(19) United States
(12) Patent Application Publication
(10) Pub. No.: US 2025/0296904 A1
(43) Pub. Date: Sep. 25, 2025

(54) CRYSTAL OF 4,4'-BIS(1,1-BIS(4-HYDROXY-3-METHYLPHENYL)ETHYL)BIPHENYL AND METHOD FOR PRODUCING THE SAME

(71) Applicant: HONSHU CHEMICAL INDUSTRY CO., LTD., Chuo-ku, Tokyo (JP)

(72) Inventors: Ryo KIMURA, Wakayama-shi (JP); Dai NISHIKAWA, Wakayama-shi (JP)

(21) Appl. No.: 18/863,608

(22) PCT Filed: May 25, 2023
(86) PCT No.: PCT/JP2023/019450

§ 371 Date: Nov. 6, 2024

(30) Foreign Application Priority Data
May 31, 2022 (JP) 2022-088664

(51) Int. Cl. C07C 39/15; C07C 37/84

(52) U.S. Cl. CPC C07C 39/15; C07C 37/84; C07B 2200/13

(57) ABSTRACT

An object is to provide an isolate of 4,4′-bis(1,1-bis(4-hydroxy-3-methylphenyl)ethyl)biphenyl in a form suitable for industrial production. As a solution, a crystal of 4,4′-bis(1,1-bis(4-hydroxy-3-methylphenyl)ethyl)biphenyl is provided.

### TECHNICAL FIELD

The present invention relates to a crystal of 4,4′-bis(1,1-bis(4-hydroxy-3-methylphenyl)ethyl)biphenyl and a method for producing the crystal.

### BACKGROUND ART

Tetrakis phenol compounds are usefully used, for example, as raw materials of epoxy resins used for sealing materials, laminating materials, electrical insulating materials, etc. for integrated circuits, curing agents for epoxy resins, developers and anti-fading agents used for thermal recording, and raw materials of electronic materials and photosensitive materials, and are widely and usefully used also as additives and clathrate compounds for antioxidants, antiseptics, antimicrobial and antifungal agents, etc.

As a method for producing a tetrakis phenol compound, for example, PTL 1 specifically describes a method in which phenol and 4,4′-diacylbiphenyl are allowed to undergo a dehydration-condensation reaction using 3-mercaptopropionic acid as a promoter in the presence of hydrogen chloride gas.

However, regarding 4,4′-bis(1,1-bis(4-hydroxy-3-methylphenyl)ethyl)biphenyl (hereinafter also referred to as “compound A”), which is one of the tetrakis phenol compounds, only its chemical name is described as a specific example of a compound in PTL 2, and no reports have yet been made on specific production methods, physical properties, etc. of compound A.

Furthermore, as a matter of course, no crystals of compound A have yet been obtained.

### CITATION LIST

### Patent Literature

PTL 1: Japanese Unexamined Patent Application Publication No. 08-027052
PTL 2: Japanese Unexamined Patent Application Publication No. 2001-235872

### SUMMARY OF INVENTION

### Technical Problem

The present invention has been made in view of the foregoing circumstances, and an object thereof is to provide an isolate of compound A in a form suitable for industrial production.

### Solution to Problem

The present inventors have conducted intensive studies on how to isolate compound A and found for the first time that compound A can be isolated as a crystal, thereby completing the present invention.

The present invention is as follows.

1. A crystal of 4,4′-bis(1,1-bis(4-hydroxy-3-methylphenyl)ethyl)biphenyl.
2. The crystal of 4,4′-bis(1,1-bis(4-hydroxy-3-methylphenyl)ethyl)biphenyl according to 1., wherein an endothermic peak top temperature determined by differential scanning calorimetry is in a range of 150° C. to 165° C.
3. The crystal of 4,4′-bis(1,1-bis(4-hydroxy-3-methylphenyl)ethyl)biphenyl according to 1., having diffraction peaks at diffraction angles 2θ of 7.8°±0.2°, 13.8°±0.2°, and 16.9°±0.2° in a powder X-ray diffraction peak pattern obtained using Cu-Kα radiation.
4. A method for producing the crystal of 4,4′-bis(1,1-bis(4-hydroxy-3-methylphenyl)ethyl)biphenyl according to 1, or 2., the method including a step of crystallizing 4,4′-bis(1,1-bis(4-hydroxy-3-methylphenyl)ethyl)biphenyl from a solution containing an alkyl acetate ester solvent having a total of 4 to 8 carbon atoms and a cyclic saturated hydrocarbon solvent having 5 to 8 carbon atoms.
5. The crystal of 4,4′-bis(1,1-bis(4-hydroxy-3-methylphenyl)ethyl)biphenyl according to 1., wherein endothermic peak top temperatures determined by differential scanning calorimetry are in a range of 120° C. to 130° C., a range of 170° C. to 185° C., and a range of 235° C. to 245° C.
6. The crystal of 4,4′-bis(1,1-bis(4-hydroxy-3-methylphenyl)ethyl)biphenyl according to 1., having diffraction peaks at diffraction angles 2θ of 11.1°±0.2° and 13.4°±0.2° in a powder X-ray diffraction peak pattern obtained using Cu-Kα radiation.
7. The crystal of 4,4′-bis(1,1-bis(4-hydroxy-3-methylphenyl)ethyl)biphenyl according to 1., having diffraction peaks at diffraction angles 2θ of 10.8°±0.2° and 16.9°±0.2° in a powder X-ray diffraction peak pattern obtained using Cu-Kα radiation.
8. A method for producing the crystal of 4,4′-bis(1,1-bis(4-hydroxy-3-methylphenyl)ethyl)biphenyl according to 1, or 5., the method including a step of crystallizing 4,4′-bis(1,1-bis(4-hydroxy-3-methylphenyl)ethyl)biphenyl from a solution containing an alkyl acetate ester solvent-based solvent having a total of 4 to 8 carbon atoms and an aromatic hydrocarbon solvent having 7 to 10 carbon atoms.

### Advantageous Effects of Invention

The crystal of compound A according to the present invention is suitable for industrial production because of having easy-to-handle properties, and can be obtained efficiently and as highly pure compound A. In addition, the crystal has a high bulk density and thus allows a large amount of compound A to be filled in a certain-volume container, thus being excellent in transportation efficiency, storage efficiency, reaction efficiency, etc.

The method for producing a crystal of compound A according to the present invention enables compound A to be isolated as a crystal easy to handle and having a high bulk density, in addition, can be a production process that is industrially feasible and efficient, and can produce highly pure compound A.

### BRIEF DESCRIPTION OF DRAWINGS

FIG. 1 shows a chart of differential scanning calorimetry (DSC) data of a crystal α of compound A obtained in Example 1.

FIG. 2 shows a chart of powder X-ray diffraction (PXRD) measurement of the crystal α of compound A obtained in Example 1.

FIG. 3 shows a chart of differential scanning calorimetry (DSC) data of a crystal α of compound A obtained in Example 2.

FIG. 4 shows a chart of powder X-ray diffraction (PXRD) measurement of the crystal α of compound A obtained in Example 2.

FIG. 5 shows a chart of differential scanning calorimetry (DSC) data of a crystal β of compound A obtained in Example 3.

FIG. 6 shows a chart of powder X-ray diffraction (PXRD) measurement of the crystal β of compound A obtained in Example 3.

FIG. 7 shows a chart of differential scanning calorimetry (DSC) data of a crystal β of compound A obtained in Example 4.

FIG. 8 shows a chart of powder X-ray diffraction (PXRD) measurement of the crystal β of compound A obtained in Example 4.

FIG. 9 shows a chart of differential scanning calorimetry (DSC) data of a crystal β of compound A obtained in Example 5.

FIG. 10 shows a chart of powder X-ray diffraction (PXRD) measurement of the crystal β of compound A obtained in Example 5.

FIG. 11 shows a chart of differential scanning calorimetry (DSC) data of a crystal β of compound A obtained in Example 6.

FIG. 12 shows a chart of powder X-ray diffraction (PXRD) measurement of the crystal β of compound A obtained in Example 6.

FIG. 13 shows a chart of differential scanning calorimetry (DSC) data of a crystal β of compound A obtained in Example 7.

FIG. 14 shows a chart of powder X-ray diffraction (PXRD) measurement of the crystal β of compound A obtained in Example 7.

FIG. 15 shows a chart of differential scanning calorimetry (DSC) data of a crystal of compound A obtained in Reference Example 1.

FIG. 16 shows a chart of powder X-ray diffraction (PXRD) measurement of the crystal of compound A obtained in Reference Example 1.

FIG. 17 shows a chart of differential scanning calorimetry (DSC) data of a crystal of compound A obtained in Reference Example 2.

FIG. 18 shows a chart of powder X-ray diffraction (PXRD) measurement of the crystal of compound A obtained in Reference Example 2.

### DESCRIPTION OF EMBODIMENTS

The present invention will be described in detail below.

4,4′-Bis(1,1-bis(4-hydroxy-3-methylphenyl)ethyl)biphenyl (compound A) according to the present invention is a compound represented by the following chemical structure.

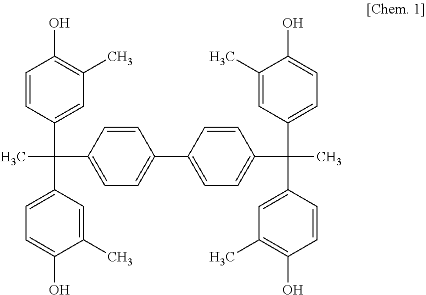 [SMILES: Cc1cc(C(C)(c2ccc(-c3ccc(C(C)(c4ccc(O)c(C)c4)c4ccc(O)c(C)c4)cc3)cc2)c2ccc(O)c(C)c2)ccc1O]

Compound A may be produced by any method, and one example is a production method in which as a result of a dehydration-condensation reaction between 4 equivalents of o-cresol and 1 equivalent of 4,4′-diacylbiphenyl, 1 equivalent of compound A and 2 equivalents of water are produced, as shown by the following reaction formula.

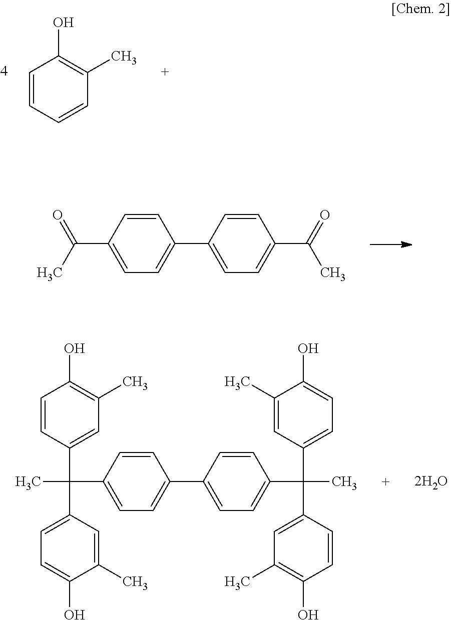

Reaction conditions in the method for producing compound A represented by the above reaction formula will be described below.

The amount of o-cresol used is preferably in the range of 4 to 20 mol, more preferably in the range of 5 to 15 mol, particularly preferably in the range of 8 to 12 mol, relative to 1 mol of 4,4′-diacylbiphenyl. If the amount of o-cresol used is less than 4 mol, the reaction proceeds slowly, and in addition, not only compound A of interest but also by-products such as a polynuclear structure resulting from further condensation of 4,4′-diacylbiphenyl and o-cresol will be formed in large amounts, which is not preferred. If o-cresol is used in an amount of more than 20 mol, the reaction rate improves, but the amount of unreacted o-cresol to be recovered increases to decrease productivity, which is not practical.

The reaction temperature is preferably in the range of 0° C. to 80° C., more preferably in the range of 30° C. to 60° C.

The reaction is typically carried out under normal pressure, but depending on the boiling point of an organic solvent used, the reaction may be carried out under increased pressure or reduced pressure so that the reaction temperature falls within the above range.

In the reaction, the method of mixing the raw materials and others is not particularly limited, but mixing a solution containing a part of o-cresol, a catalyst, and if necessary a promoter with a mixed solution of 4,4′-diacetylbiphenyl and the rest of o-cresol is preferred from the viewpoint of reaction selectivity. In the case of this method of mixing, the time required for mixing is in the range of 0.5 to 5 hours. The reaction is carried out such that the solution after mixing satisfies the above amount of raw materials used.

Although depending on the amount of catalyst and the reaction temperature, the reaction time is typically in the range of 1 to 20 hours, and the reaction is preferably completed in the range of 1 to 10 hours.

The endpoint of the reaction can be determined by liquid chromatography analysis or gas chromatography analysis. The endpoint of the reaction is preferably defined as the time point at which unreacted 4,4′-diacylbiphenyl has disappeared or the increase of compound A of interest has no longer been observed.

### <Catalyst>

In the method for producing compound A represented by the above reaction formula, an acid catalyst, whether an inorganic acid or an organic acid, can be used as a catalyst. Examples of inorganic acids include hydrogen chloride gas, hydrochloric acid, sulfuric acid, phosphoric acid, and sulfuric anhydride, and examples of organic acids include aromatic sulfonic acids such as benzenesulfonic acid and p-toluenesulfonic acid, alkanesulfonic acids having 1 to 4 carbon atoms, such as methanesulfonic acid and ethanesulfonic acid, trifluoromethanesulfonic acid, and trichloroacetic acid. Alternatively, for example, a metal halide such as aluminum chloride or iron chloride or a solid acid such as cation-exchange resin can be used as a catalyst.

Among them, organic acids are suitable for use. Among the organic acids, alkanesulfonic acids having 1 to 4 carbon atoms are more preferred, methanesulfonic acid or ethanesulfonic acid is still more preferred, and methanesulfonic acid is particularly preferred. The amount of the alkanesulfonic acid used is preferably in the range of 0.1 to 5.0 mol, more preferably in the range of 0.5 to 2.0 mol, relative to 1 mol of 4,4′-diacylbiphenyl.

### <Promoter>

In the method for producing compound A represented by the above reaction formula, a thiol compound may be used as a promoter in combination with the catalyst, if necessary. The thiol compound is not particularly limited as long as it is a compound having a mercapto group and it does not adversely affect reaction selectivity, etc. Examples of such compounds include carboxylic acids having a mercapto group, such as 3-mercaptopropionic acid and thioglycolic acid, alkyl mercaptans having 1 to 12 carbon atoms, such as methyl mercaptan, 1-octanethiol (octyl mercaptan), and 1-dodecanethiol (lauryl mercaptan), and mercapto alcohols such as mercaptoethanol and mercaptobutanol. In particular, alkyl mercaptans having 1 to 12 carbon atoms, such as 1-octanethiol, are suitable. They may also be used in the form of an aqueous solution of a sodium salt.

When a thiol compound is used, the amount used is preferably in the range of 1 to 10 wt % relative to the amount of 4,4′-diacylbiphenyl. If the amount used is less than 1 wt %, the thiol compound cannot sufficiently function as a promoter, and even if the amount used is more than 10 wt %, the thiol compound cannot function more effectively as a promoter, and the selectivity is almost the same.

### <Reaction Solvent>

In performing the method for producing compound A represented by the above reaction formula, it is not necessary to use a reaction solvent if there is no problem with operability. However, the reaction solvent may be used for better operability during industrial production. The reaction solvent for use is not particularly limited as long as it is a solvent that does not distill from a reaction vessel at the reaction temperature and is inert to the reaction. Examples include aromatic hydrocarbons such as toluene, xylene, and benzene, halogenated aromatic hydrocarbons such as chlorobenzene and dichlorobenzene, aliphatic hydrocarbons such as pentane, n-hexane, cyclohexane, and heptane, aliphatic alcohols such as methanol, n-butanol, t-butanol, and cyclohexanol, and aliphatic or cyclic ethers such as diethyl ether, diisopropyl ether, methyl-t-butyl ether, diphenyl ether, tetrahydrofuran, and dioxane. Among the reaction solvents, aromatic hydrocarbons and halogenated aromatic hydrocarbons are preferred, and aromatic hydrocarbons are more preferred. The amount of the reaction solvent used is not particularly limited, but from the viewpoint of economic efficiency, it is typically in the range of 0.1 to 10 times, preferably in the range of 0.1 to 2 times, more preferably in the range of 0.1 to 1 time the amount of o-cresol on a weight basis.

In the method for producing compound A represented by the above reaction formula, water is produced as a result of the dehydration-condensation reaction between o-cresol and 4,4′-diacylbiphenyl. The reaction is preferably carried out under dehydrating conditions where water in the reaction system, such as the reaction product water and water contained in the catalyst used, can be removed, because the reaction proceeds more rapidly than when dehydration is not performed, and the formation of by-products is suppressed, so that the target can be obtained in higher yield. Examples of methods of dehydration include, but are not limited to, dehydration by addition of a dehydrating agent, dehydration by pressure reduction, and dehydration by azeotropy with a solvent under normal pressure or under reduced pressure. Examples of dehydrating agents that can be added as required include, but are not limited to, organic dehydrating agents having an orthoester skeleton, such as methyl orthoformate, ethyl orthoformate, methyl orthoacetate, ethyl orthopropionate, methyl ortho-n-butyrate, methyl ortho-i-butyrate, and 1,1,1-trimethoxyoctane, zeolites such as molecular sieve (3A) and molecular sieve (4A), and inorganic anhydrous salts capable of containing crystal water in molecules, such as calcium chloride (anhydrous), calcium sulfate (anhydrous), magnesium chloride (anhydrous), magnesium sulfate (anhydrous), potassium carbonate (anhydrous), potassium sulfide (anhydrous), potassium subsulfide (anhydrous), sodium sulfate (anhydrous), sodium sulfite (anhydrous), and copper sulfate (anhydrous).

### <Treatment After Completion of Reaction>

Methods of post-treatments in the method for producing compound A represented by the above reaction formula will be described below.

The resulting final reaction mixture is preferably subjected to post-treatments before a crystallization method of the present invention described later is performed. Examples of the post-treatments include neutralizing the acid catalyst used by adding alkaline water such as an aqueous sodium hydroxide solution to the final reaction mixture, washing an oil phase containing compound A with water optionally after adding a solvent that dissolves compound A and that is separable from water, such as toluene or xylene, and removing the solvent and o-cresol excessively used in the reaction by distillation.

As compound A to be subjected to the crystallization method of the present invention described later, for example, a distillation residue containing compound A obtained by removing the solvent and o-cresol from the reaction mixture by distillation as described above, compound A subjected to column separation, a crystal obtained by the crystallization method according to the present invention, or a crystal obtained by a crystallization method other than the crystallization method according to the present invention can be used.

### <Crystal of Present Invention: Crystal α>

One of the crystals of compound A in the present invention is a crystal (hereinafter referred to as a “crystal α”) having one or both characteristics of “the endothermic peak top temperature” and “the powder X-ray diffraction peak pattern”, that is, having an endothermic peak top temperature in the range of 150° C. to 165° C. as determined by differential scanning calorimetry and having diffraction peaks at diffraction angles 2θ of 7.8°±0.2°, 13.8°±0.2°, and 16.9°±0.2° in a powder X-ray diffraction peak pattern obtained using Cu-Kα radiation.

The endothermic peak top temperature of the crystal α determined by differential scanning calorimetry is preferably in the range of 152° C. to 165° C., more preferably in the range of 154° C. to 165° C., particularly preferably in the range of 155° C. to 163° C.

In addition to the endothermic peak top temperature of the crystal α determined by differential scanning calorimetry in the above range, there may be an endothermic peak top temperature in the range of 237° C. to 245° C. The peak top temperature of this endothermic peak is more preferably in the range of 238° C. to 245° C., particularly preferably in the range of 238° C. to 243° C.

In the powder X-ray diffraction peak pattern obtained using Cu-Kα radiation, the crystal α preferably has diffraction peaks at diffraction angles 2θ of 12.1°±0.2° and 13.0°±0.2° in addition to the above diffraction peaks, more preferably has further additional diffraction peaks at 20.9°±0.2° and 21.7°±0.2°, particularly preferably has further additional diffraction peaks at 15.5°±0.2° and 17.8°±0.2°.

The purity of compound A in the crystal α is preferably 97.0 area % or more, more preferably 97.5 area % or more, still more preferably 98.0 area % or more. The value of the purity of compound A is calculated by the following formula using values of the total peak area, the peak area of compound A, and the peak area of various solvents, the areas being observed when a sample of a methanol solution of the crystal α at a concentration in the range of 25 to 35 mg/50 mL is prepared and analyzed by ultra-high-performance liquid chromatography.

The above ultra-high-performance liquid chromatography analysis is meant to be carried out with the following analytical instrument and analytical conditions or with an analytical instrument and analytical conditions equivalent thereto.

$$“ Purity of compound A ” = ( “ peak area of compound A ” \div “ total peak area ” ) \times \{ 100 \% \div ( 100 \% - “ area \% of various solvents ” ) \} \qquad [ Calculation formula ]$$

### <Instrument and Conditions>

Apparatus: Shimadzu UFLC LC-20 series/manufactured by Shimadzu Corporation

Pump: LC-20AD

Column oven: CTO-20A

Detector: SPD-20A

Column: HALO C18, 3.0×75 mm/manufactured by Shimadzu GLC Ltd.

Oven temperature: 50° C.

Flow rate: 0.7 mL/min

Mobile phase: (A) 0.2 vol % aqueous acetic acid solution, (B) methanol

Gradient conditions: (B) vol % (time from start of analysis)

50% (0 min)→(10 min)→100% (15 min)

Sample injection volume: 1 to 5 μL

Detection wavelength: 280 nm

The loose bulk density of the crystal α is preferably in the range of 0.25 to 0.40 g/cm³, more preferably in the range of 0.28 to 0.40 g/cm³, still more preferably in the range of 0.30 to 0.40 g/cm³. The value of this loose bulk density means a numerical value calculated by dividing the value of the weight of a weighed crystal by the value of the volume measured immediately after the weighed crystal is filled in a graduated cylinder.

The tapped bulk density of the crystal α is preferably in the range of 0.40 to 0.55 g/cm³, more preferably in the range of 0.42 to 0.55 g/cm³, still more preferably in the range of 0.45 to 0.55 g/cm³. The value of this tapped bulk density means a numerical value calculated by dividing the value of the weight of a weighed crystal by the value of the volume measured after the weighed crystal is filled in a graduated cylinder and then manually vibrated 300 times.

### <Crystal of Present Invention: Crystal β>

Another one of the crystals of compound A in the present invention is a crystal (hereinafter referred to as a “crystal β”) having one or both characteristics of “the endothermic peak top temperature” and “the powder X-ray diffraction peak pattern (pattern 1 or pattern 2)”, that is, having endothermic peak top temperatures in the range of 120° C. to 130° C., the range of 170° C. to 185° C., and the range of 235° C. to 245° C. as determined by differential scanning calorimetry and having diffraction peaks at diffraction angles 2θ of 11.1°±0.2° and 13.4°±0.2° in a powder X-ray diffraction peak pattern obtained using Cu-Kα radiation (pattern 1) or having diffraction peaks at diffraction angles 2θ of 10.8°±0.2° and 16.9°±0.2° (pattern 2).

The endothermic peak top temperatures of the crystal β determined by differential scanning calorimetry are preferably in the range of 122° C. to 130° C., the range of 172° C. to 185° C., and the range of 237° C. to 245° C., more preferably in the range of 123° C. to 130° C., the range of 174° C. to 183° C., and the range of 238° C. to 245° C., particularly preferably in the range of 123° C. to 128° C., the range of 175° C. to 180° C., and the range of 238° C. to 243° C.

Regarding the powder X-ray diffraction peak pattern of the crystal β obtained using Cu-Kα radiation, in the case of pattern 1, the crystal β preferably has diffraction peaks at diffraction angles 2θ of 16.0°±0.2° and 16.4°±0.2° in addition to the above diffraction peaks, more preferably has further additional diffraction peaks at diffraction angles 20 of 18.00±0.2°, 19.30±0.2°, 20.00±0.2°, and 22.6°±0.2°.

Regarding the powder X-ray diffraction peak pattern of the crystal β obtained using Cu-Kα radiation, in the case of pattern 2, the crystal β preferably has diffraction peaks at diffraction angles 2θ of 13.2°±0.2°, 13.6°±0.2°, 14.0°±0.2°, and 23.6°±0.2° in addition to the above diffraction peaks, more preferably has further additional diffraction peaks at diffraction angles 2θ of 15.9°±0.2°, 20.6°±0.2°, and 21.6°±0.2°.

The purity of compound A in the crystal β is preferably 97.0 area % or more, more preferably 97.5 areas or more, still more preferably 98.0 areas or more. The value of the purity of compound A is calculated by the following formula using values of the total peak area, the peak area of compound A, and the peak area of various solvents, the areas being observed when a sample of a methanol solution of the crystal β at a concentration in the range of 25 to 35 mg/50 mL is prepared and analyzed by ultra-high-performance liquid chromatography.

The above ultra-high-performance liquid chromatography analysis is meant to be carried out with the following analytical instrument and analytical conditions or with an analytical instrument and analytical conditions equivalent thereto.

$$“ Purity of compound A ” = ( “ peak area of compound A ” \div “ total peak area ” ) \times \{ 100 \% \div ( 100 \% - “ area \% of various solvents ” ) \} \qquad [ Calculation formula ]$$

### <Instrument and Conditions>

Apparatus: Shimadzu UFLC LC-20 series/manufactured by Shimadzu Corporation

Pump: LC-20AD

Column oven: CTO-20A

Detector: SPD-20A

Column: HALO C18, 3.0×75 mm/manufactured by Shimadzu GLC Ltd.

Oven temperature: 50° C.

Flow rate: 0.7 mL/min

Mobile phase: (A) 0.2 vol % aqueous acetic acid solution, (B) methanol

Gradient conditions: (B) vol % (time from start of analysis)

50% (0 min)→(10 min)→100% (15 min)

Sample injection volume: 1 to 5 μL

Detection wavelength: 280 nm

The loose bulk density of the crystal β is preferably in the range of 0.25 to 0.40 g/cm³, more preferably in the range of 0.28 to 0.40 g/cm³, still more preferably in the range of 0.30 to 0.40 g/cm³. The value of this loose bulk density means a numerical value calculated by dividing, by the value of the volume measured immediately after a crystal is filled in a graduated cylinder, the value of the weight of the filled crystal.

The tapped bulk density of the crystal β is preferably in the range of 0.40 to 0.55 g/cm³, more preferably in the range of 0.42 to 0.55 g/cm³, still more preferably in the range of 0.45 to 0.55 g/cm³. The value of this tapped bulk density means a numerical value calculated by dividing, by the value of the volume measured after a crystal is filled in a graduated cylinder and then manually vibrated 300 times, the value of the weight of the filled crystal.

The present inventors presume that the crystal α having diffraction peaks at diffraction angles 2θ of 7.8°±0.2°, 13.8°±0.2°, and 16.9°±0.2° in a powder X-ray diffraction peak pattern obtained using Cu-Kα radiation is a crystal in which cyclohexane is solvated because it is obtained by crystallization using a solution containing cyclohexane and contains cyclohexane also after being dried.

Regarding the fact that the crystal α sometimes exhibits two endothermic peaks in differential scanning calorimetry, the present inventors presume that a peak appearing in the range of 150° C. to 165° C. indicates that the crystal is melted, and a peak appearing in the range of 237° C. to 245° C. indicates that the crystal recrystallized during differential scanning calorimetry is melted. It has also been observed that a behavior indicating heat generation sometimes appears between the peak appearing in the range of 150° C. to 165° C. and the peak appearing in the range of 237° C. to 245° C. From this, it is presumed that the crystal α is a crystal including one kind of crystal phase and is not a mixed crystal of a plurality of different crystal phases.

Regarding the fact that the crystal β exhibits three endothermic peaks in differential scanning calorimetry, the present inventors presume that a peak appearing in the range of 120° C. to 130° C. indicates that an aromatic hydrocarbon solvent having 7 to 10 carbon atoms solvated in the crystal is eliminated, a peak appearing in the range of 170° C. to 185° C. indicates that the crystal is melted, and a peak appearing in the range of 235° C. to 245° C. indicates that the crystal recrystallized during differential scanning calorimetry is melted. It has also been observed that a behavior indicating heat generation sometimes appears between the peak appearing in the range of 170° C. to 185° C. and the peak appearing in the range of 235° C. to 245° C. From this, it is presumed that the crystal β is a crystal including one kind of crystal phase and is not a mixed crystal of a plurality of different crystal phases.

In addition, the present inventors presume that the crystal β having diffraction peaks at diffraction angles 20 of 11.1°±0.2° and 13.4°±0.2° in a powder X-ray diffraction peak pattern obtained using Cu-Kα radiation (pattern 1) is a crystal in which toluene is solvated because it is obtained by crystallization using a solution containing toluene, contains toluene also after being dried, and exhibits, in differential scanning calorimetry, an endothermic peak in addition to the endothermic peak observed when the crystal melts.

Furthermore, the present inventors presume that the crystal β having diffraction peaks at diffraction angles 20 of 10.8°±0.2° and 16.9°±0.2° in a powder X-ray diffraction peak pattern obtained using Cu-Kα radiation (pattern 2) is a crystal in which ethylbenzene is solvated because it is obtained by crystallization using a solution containing ethylbenzene, contains ethylbenzene also after being dried, and exhibits, in differential scanning calorimetry, an endothermic peak in addition to the endothermic peak observed when the crystal melts.

### <Method for Producing Crystal α>

The crystal α can be produced by performing a step of crystallizing compound A from a solution containing an alkyl acetate ester solvent having a total of 4 to 8 carbon atoms and a cyclic saturated hydrocarbon solvent having 5 to 8 carbon atoms.

Specific examples of the alkyl acetate ester solvent having a total of 4 to 8 carbon atoms include ethyl acetate (a total of 4 carbon atoms), butyl acetate (a total of 6 carbon atoms), amyl acetate (a total of 7 carbon atoms), isoamyl acetate (a total of 7 carbon atoms), 2-methylbutyl acetate (a total of 7 carbon atoms), and n-hexyl acetate (a total of 8 carbon atoms). Of these, butyl acetate is particularly preferred.

Specific examples of the cyclic saturated hydrocarbon solvent having 5 to 8 carbon atoms include cyclopentane and cyclohexane. Of these, cyclohexane is particularly preferred.

Among the crystals α, to obtain a crystal α having diffraction peaks at diffraction angles 2θ of 7.8°±0.2°, 13.8°±0.2°, and 16.9°±0.2° in a powder X-ray diffraction peak pattern obtained using Cu-Kα radiation, cyclohexane is used as the cyclic saturated hydrocarbon solvent having 5 to 8 carbon atoms.

For the amounts of these solvents used, the amount of the alkyl acetate ester solvent having a total of 4 to 8 carbon atoms is preferably in the range of 0.5 to 3 times, more preferably in the range of 0.5 to 2 times, still more preferably in the range of 0.75 to 1.5 times, particularly preferably in the range of 0.75 to 1.25 times the amount of compound A used on a weight basis. The amount of the cyclic saturated hydrocarbon solvent having 5 to 8 carbon atoms is preferably in the range of 1 to 5 times, more preferably in the range of 1 to 3 times, particularly preferably in the range of 1 to 2 times the amount of compound A used on a weight basis.

In this crystallization step, the crystal begins to be precipitated at a crystallization solution temperature preferably in the range of 50° C. to 100° C., more preferably in the range of 60° C. to 90° C., particularly preferably in the range of 65° C. to 85° C. The cooling rate in cooling the crystallization solution in the crystallization step is preferably in the range of 5° C. to 20° C. per hour, more preferably in the range of 7° C. to 15° C. per hour, still more preferably in the range of 7° C. to 12° C. per hour. The final crystallization solution temperature in the crystallization step is preferably in the range of 10° C. to 30° C.

In precipitating the crystal, it is not necessary to use a seed crystal, but it is preferable to use a seed crystal. Any crystal may be used as a seed crystal, but it is preferable to use the crystal α. The seed crystal of the crystal α may be prepared by performing the crystallization step without a seed crystal. The amount of the seed crystal used is preferably in the range of 0.1 to 1.0 wt % relative to the amount of compound A. The precipitated crystal can be separated by, for example, a filtration operation such as centrifugal filtration to thereby isolate the crystal.

### <Method for Producing Crystal β>

The crystal β can be produced by performing a step of crystallizing compound A from a solution containing an alkyl acetate ester solvent having a total of 4 to 8 carbon atoms and an aromatic hydrocarbon solvent having 7 to 10 carbon atoms.

Specific examples of the alkyl acetate ester solvent having a total of 4 to 8 carbon atoms include ethyl acetate (a total of 4 carbon atoms), butyl acetate (a total of 6 carbon atoms), amyl acetate (a total of 7 carbon atoms), isoamyl acetate (a total of 7 carbon atoms), 2-methylbutyl acetate (a total of 7 carbon atoms), and n-hexyl acetate (a total of 8 carbon atoms). Of these, butyl acetate is particularly preferred.

Specific examples of the aromatic hydrocarbon solvent having 7 to 10 carbon atoms include toluene, ethylbenzene, xylene, and trimethine. Of these, toluene or ethylbenzene is particularly preferred.

Among the crystals β, to obtain a crystal β having diffraction peaks at diffraction angles 2θ of 11.1°±0.2° and 13.4°±0.2° in a powder X-ray diffraction peak pattern obtained using Cu-Kα radiation (pattern 1), toluene is used as the aromatic hydrocarbon solvent having 7 to 10 carbon atoms.

Among the crystals β, to obtain a crystal β having diffraction peaks at diffraction angles 2θ of 10.8°±0.2° and 16.9°±0.2° in a powder X-ray diffraction peak pattern obtained using Cu-Kα radiation (pattern 2), ethylbenzene is used as the aromatic hydrocarbon solvent having 7 to 10 carbon atoms.

For the amounts of these solvents used, the amount of the alkyl acetate ester solvent having a total of 4 to 8 carbon atoms is preferably in the range of 0.5 to 2 times, more preferably in the range of 0.5 to 1.5 times, particularly preferably in the range of 0.75 to 1.25 times the amount of compound A used on a weight basis. The amount of the cyclic saturated hydrocarbon solvent having 5 to 8 carbon atoms is preferably in the range of 1 to 5 times, more preferably in the range of 1 to 3 times, particularly preferably in the range of 1 to 2 times the amount of compound A used on a weight basis.

In this crystallization step, the crystal begins to be precipitated at a crystallization solution temperature preferably in the range of 50° C. to 100° C., more preferably in the range of 60° C. to 90° C., particularly preferably in the range of 65° C. to 85° C. The cooling rate in cooling the crystallization solution in the crystallization step is preferably in the range of 5° C. to 20° C. per hour, more preferably in the range of 7° C. to 15° C. per hour, still more preferably in the range of 7° C. to 12° C. per hour. The final crystallization solution temperature in the crystallization step is preferably 10° C. to 30° C.

In precipitating the crystal, it is not necessary to use a seed crystal, but it is preferable to use a seed crystal. Any crystal may be used as a seed crystal, but it is preferable to use the crystal β. The seed crystal of the crystal β may be prepared by performing the crystallization step without a seed crystal. The amount of the seed crystal used is preferably in the range of 0.1 to 1.0 wt % relative to the amount of compound A. The precipitated crystal can be separated by, for example, a filtration operation such as centrifugal filtration to thereby isolate the crystal.

The crystal is preferably further washed with a solvent. As the solvent used here, a cyclic saturated hydrocarbon solvent having 5 to 8 carbon atoms is preferred in the case of the crystal α, and an aromatic hydrocarbon solvent having 7 to 10 carbon atoms is preferred in the case of the crystal β. The amount of the solvent used is preferably in the range of 0.5 to 5 times, more preferably in the range of 0.5 to 2.5 times, still more preferably in the range of 0.5 to 2 times, particularly preferably 1 time the amount of the obtained crystal on a weight basis.

The obtained crystal can be dried to thereby remove the solvent used. The drying operation may be performed at a temperature preferably in the range of 20° C. to 100° C., more preferably in the range of 65° C. to 95° C., still more preferably 80° C. The drying may be performed under normal pressure or reduced pressure, but in the case of industrial practice, a reduced pressure of about 10 kPa is preferred, a reduced pressure of about 5 kPa is more preferred, and a reduced pressure of about 1.5 kPa is still more preferred. Performing the drying under such a reduced pressure is preferred because the solvent used can be removed more efficiently.

### EXAMPLES

The present invention will now be described specifically with reference to Examples and Reference Examples, but these Examples and Reference Examples are not intended to limit the present invention.

### (1) Measurement of Purity and Solvent Content of Compound A in Crystal

The purity of compound A in crystals obtained in Examples and Reference Examples and the amount of solvent contained in the crystals were measured under the following measurement conditions.

### Measurement Conditions (A)

### Ultra-High-Performance Liquid Chromatography

Apparatus: Shimadzu UFLC LC-20 series/manufactured by Shimadzu Corporation

Pump: LC-20AD

Column oven: CTO-20A

Detector: SPD-20A

Column: HALO C18, 3.0×75 mm/manufactured by Shimadzu GLC Ltd.

Oven temperature: 50° C.

Flow rate: 0.7 mL/min

Mobile phase: (A) 0.2 vol % aqueous acetic acid solution, (B) methanol

Gradient conditions: (B) vol % (time from start of analysis)

50% (0 min)→(10 min)→100% (15 min)

Sample injection volume: 1 to 5 μL

Detection wavelength: 280 nm, 254 nm

The purity of compound A in an obtained crystal was calculated by the following formula using values of the total peak area, the peak area of compound A, and the peak area of various solvents, the areas being observed at a detection wavelength of 280 nm when a sample of a methanol solution of the crystal at a concentration in the range of 25 to 35 mg/50 mL was prepared and measured under the measurement conditions (A).

$$“ Purity of compound A ” = ( “ peak area of compound A ” \div “ total peak area ” ) \times \{ 100 \% \div ( 100 \% - “ area \% of various solvents ” ) \} \qquad [ Calculation formula ]$$

### Measurement Conditions (B)

### Liquid Chromatography

Apparatus: Shimadzu HPLC LC-20 series/manufactured by

Shimadzu Corporation

Pump: LC-20AT

Column oven: CTO-20A

Detector: SPD-20A

Column: Shim-pack OLC-ODS, 6 mm×15 cm/manufactured by Shimadzu GLC Ltd.

Oven temperature: 50° C.

Flow rate: 1.0 mL/min

Mobile phase: (A) 0.1 vol % aqueous phosphoric acid solution, (B) acetonitrile

Gradient conditions: (B) vol % (time from start of analysis)

50% (0 min)→(20 min)→100% (27 min)

Sample injection volume: 20 μL

Detection wavelength: 280 nm, 210 nm

### Measurement Conditions (C)

### Gas Chromatography

Apparatus: GC-2010 Plus/manufactured by Shimadzu Corporation

Detection type: FID

Column: TC-160m manufactured by GL Sciences Inc.; inner diameter, 0.25 mm

Film thickness: 0.25 μm

Carrier gas: nitrogen

Column temperature: 40° C. (0 min)→40° C. (10 min)→20° C./min→300° C. (5 min)

Vaporization chamber temperature: 300° C.

Control mode: pressure

Total flow rate: 8.5 mL/min

Column flow rate: 0.92 mL/min

Linear velocity: 19.9 cm/s

Purge flow rate: 3.0 mL/min

Split ratio: 5.0

Detector temperature: 310° C.

Makeup flow rate: 30.0 mL/min

Hydrogen flow rate: 40.0 mL/min

Air flow rate: 400.0 mL/min

### HS Sampler

Apparatus: TurboMatrix HS40/PerkinElmer Inc.

Oven temperature: 100° C.

Needle temperature: 105° C.

Transfer temperature: 105° C.

Temperature holding time: 20 min

Pressing time: 3.0 min

Withdrawal time: 0.5 min

Injection time: 0.05 min

GC cycle time: 44.0 min

HS carrier gas pressure: 154.0 kPa

Headspace mode: constant

Vial venting: OFF

The reaction yield was calculated by subtracting the amounts of various solvents measured under the measurement conditions (A) to (C) from the yield of the crystal.

### (2) Differential Scanning Calorimetry

The crystals of compound A obtained in Examples and Reference Examples were inspected for the occurrence of temperature-induced exothermic/endothermic events with the following differential scanning calorimeter (DSC) under the following measurement conditions.

### [Differential Scanning Calorimeter (DSC)]

Apparatus: DSC7020/manufactured by Hitachi High-Tech Science Corporation

### [Measurement Conditions]

Heating rate: 10° C./min

Measurement temperature range: 30° C. to 300° C.

Measurement atmosphere: nitrogen, 50 mL/min

Measurement sample: crystal, 3 to 4 mg

### (3) Powder X-Ray Diffraction (PXRD) Measurement

Each of the crystals of compound A obtained in Examples and Reference Examples in an amount of 0.1 g was loaded in a sample loading section of a glass test plate and subjected to a powder X-ray diffraction measurement using the following measuring apparatus under the following measurement conditions.

### [Measuring Apparatus]

MiniFlex600-C/manufactured by Rigaku Corporation

### [Measurement Conditions]

X-ray source: CuKα

Tube voltage: 40 kV

Tube current: 15 mA

Scan axis: 2θ/θ

Mode: continuous

Measurement range: 2θ=5° to 90° Step: 0.02°

Speed measurement time: 10°/min

Entrance slit: 0.25° Receiving slit: 13.00 mm

### (4) Bulk Density Measurement

The value of the loose bulk density of each of the crystals obtained in Examples and Reference Examples was calculated by dividing, by the value of the volume measured immediately after the crystal was filled in a graduated cylinder, the value of the weight of the filled crystal.

The value of the tapped bulk density of each of the crystals obtained in Examples and Reference Examples was calculated by dividing, by the value of the volume measured after the crystal was filled in a graduated cylinder and then manually vibrated 300 times, the value of the weight of the filled crystal.

### Example 1

To a 2 L four-necked flask equipped with a stirrer, o-cresol (69.6 g), methanesulfonic acid (58.1 g), and dodecyl mercaptan (4.1 g) were added, and after nitrogen purging was performed, the temperature was raised to 50° C. While the temperature in the flask was maintained at 50° C., a solution of o-cresol (307.7 g), 4,4′-diacetylbiphenyl (79.9 g), and ethylbenzene (85.3 g) adjusted to 50° C. was mixed with stirring over 5 hours. Thereafter, stirring was performed for 22 hours. After a 16% aqueous sodium hydroxide solution (167.8 g) and a 75% aqueous phosphoric acid solution (9.7 g) were added to achieve partial neutralization, the temperature was raised to 75° C. with butyl acetate (428.0 g) added, and an aqueous layer was removed.

After water (211.9 g) was added into the flask to wash an oil layer with water, an aqueous layer was removed. Reduced-pressure distillation was performed with heating, and o-cresol, butyl acetate, and water were recovered until the distillation residue weighed 256.3 g. Thereafter, butyl acetate (213.2 g) as a crystallization solvent 1 was added to prepare a preparation solution A.

A portion (61.5 g) of the preparation solution A was put in a 300 mL four-necked flask equipped with a stirrer, and after the liquid temperature was adjusted to 74° C., cyclohexane (28.2 g) as a crystallization solvent 2 was added dropwise over 20 minutes. At this time, the liquid temperature decreased to 70° C. After 35 minutes elapsed while the temperature was maintained at 74° C., cyclohexane (14.3 g) was further added dropwise over 15 minutes. After the resultant was held at 73° C. for 1 hour, the liquid temperature was lowered to 50° C. at a rate of 10° C./h, after which the resultant was allowed to cool to 25° C. At this time, the precipitated crystal hardly adhered to the flask wall surface and was uniformly dispersed in a crystallization solution.

The precipitated crystal was separated by filtration using a centrifugal filter and washed with cyclohexane (26.7 g). The obtained crystal was transferred to a 200 mL eggplant-shaped flask, and dried at 80° C. for 3 hours under a reduced pressure of 1.5 kPa to obtain a dried crystal of compound A.

The purity of compound A in the obtained crystal was 98.1 area %, and the yield was 68% in terms of 4,4′-diacetylbiphenyl.

The obtained crystal contained 0.8 wt % o-cresol, 2.4 wt % butyl acetate, and 7.4 wt % cyclohexane.

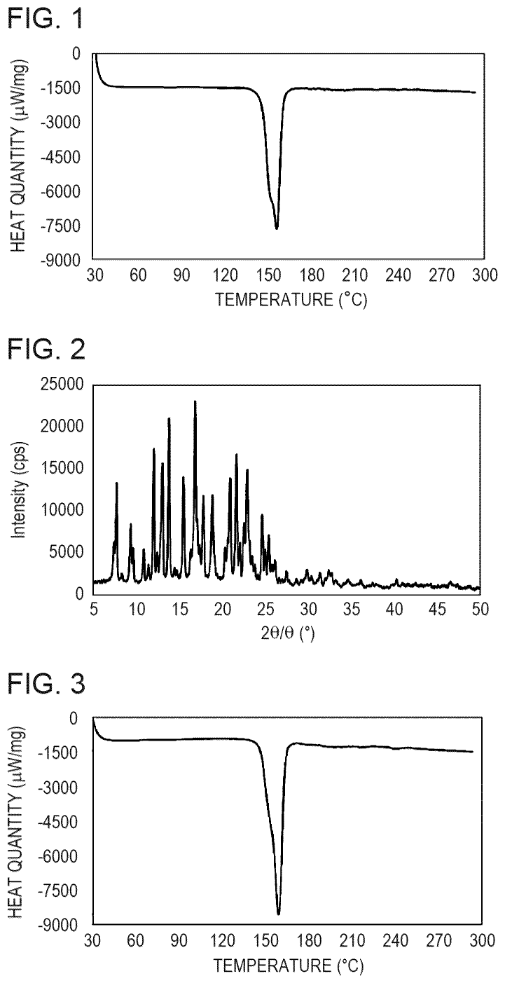

As a result of differential scanning calorimetry (DSC) of the obtained crystal, it was confirmed from an endothermic peak top temperature that the crystal had a melting point of 157° C. and was a crystal α. The DSC data are shown in FIG. 1.

Diffraction angles 2θ (°) of diffraction peaks appearing in a powder X-ray diffraction (PXRD) measurement of the obtained crystal and peaks whose relative intensity based on a peak with the greatest intensity (measured intensity) is 20 or more are shown in Table 1. The PXRD measurement chart is shown in FIG. 2.

TABLE 1

| | Relative |
|---|---|
| 2θ (°) | intensity |
| 7.5 | 27 |
| 7.8 | 58 |
| 9.4 | 37 |
| 9.6 | 24 |
| 10.9 | 24 |
| 12.1 | 75 |
| 12.4 | 22 |
| 13.0 | 68 |
| 13.8 | 91 |
| 15.5 | 60 |
| 16.4 | 24 |
| 16.9 | 100 |
| 17.4 | 26 |
| 17.8 | 51 |
| 18.9 | 51 |
| 20.3 | 24 |
| 20.9 | 60 |
| 21.7 | 73 |
| 22.1 | 27 |
| 22.5 | 37 |
| 22.9 | 64 |
| 23.4 | 20 |
| 24.6 | 41 |
| 25.0 | 24 |
| 25.4 | 31 |

### Example 2

A portion (61.9 g) of the preparation solution A prepared in Example 1 was put in a 300 mL four-necked flask equipped with a stirrer. After the liquid temperature was adjusted to 74° C., cyclohexane (28.4 g) as a crystallization solvent 2 was added dropwise over 20 minutes. At this time, the liquid temperature decreased to 71° C. After 15 minutes elapsed while the temperature was maintained at 73° C., 80 mg of the crystal α of compound A obtained in Example 1 was added as a seed crystal. After 20 minutes elapsed while the temperature was maintained at 73° C. after the addition, cyclohexane (14.4 g) was further added dropwise over 15 minutes. After 10 minutes, 80 mg of the crystal was further added. One hour after the addition, the liquid temperature was lowered to 50° C. at a rate of 10° C./h, after which the resultant was allowed to cool to 25° C. At this time, the precipitated crystal hardly adhered to the flask wall surface and was uniformly dispersed in a crystallization solution.

The precipitated crystal was separated by filtration using a centrifugal filter and washed with cyclohexane (29.0 g). The obtained crystal was transferred to a 200 mL eggplant-shaped flask, and dried at 80° C. for 3 hours under a reduced pressure of 1.5 kPa to obtain a dried crystal of compound A.

The purity of compound A in the obtained crystal was 98.5 area %, and the yield was 65% in terms of 4,4′-diacetylbiphenyl.

The obtained crystal contained 0.4 wt % o-cresol, 2.2 wt % butyl acetate, and 7.8 wt % cyclohexane.

As a result of DSC analysis of the obtained crystal, it was confirmed from an endothermic peak top temperature that the crystal had a melting point of 159° C. and was a crystal α. The DSC data are shown in FIG. 3.

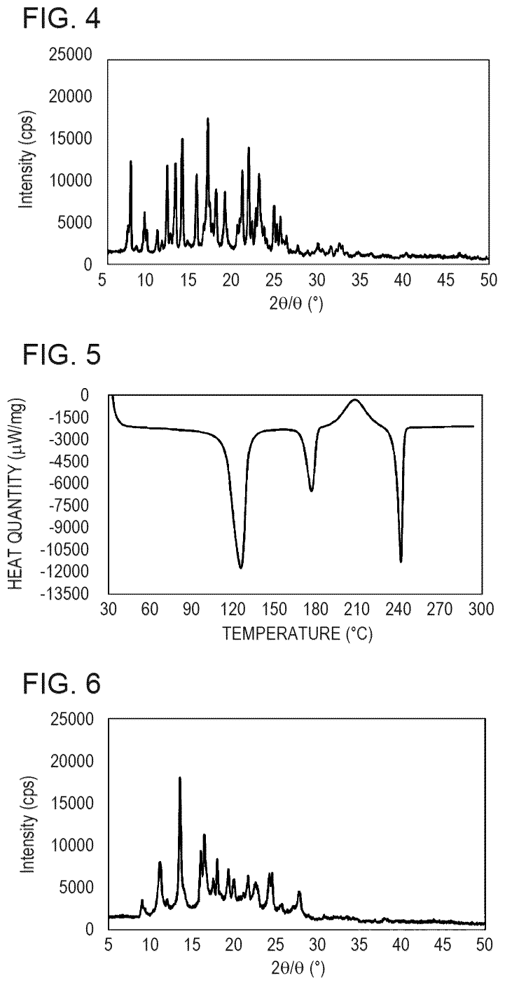

Diffraction angles 2θ (°) and relative intensities of diffraction peaks appearing in a PXRD measurement of the obtained crystal are shown in Table 2 as in Example 1. The PXRD measurement chart is shown in FIG. 4. These measurement data reveal that the crystal belongs to the same crystal system as the crystal obtained in Example 1.

TABLE 2

| | Relative |
|---|---|
| 2θ (°) | intensity |
| 7.4 | 27 |
| 7.8 | 71 |
| 9.3 | 36 |
| 9.6 | 23 |
| 10.8 | 23 |
| 12.0 | 68 |
| 12.4 | 21 |
| 13.0 | 69 |
| 13.8 | 86 |
| 15.5 | 62 |
| 16.3 | 28 |
| 16.8 | 100 |
| 17.4 | 28 |
| 17.8 | 51 |
| 18.8 | 50 |
| 20.3 | 27 |
| 20.9 | 65 |
| 21.6 | 80 |
| 22.0 | 30 |
| 22.5 | 38 |
| 22.8 | 62 |
| 23.5 | 26 |
| 24.6 | 40 |
| 25.0 | 28 |
| 25.4 | 33 |

### Example 3

A portion (63.4 g) of the preparation solution A prepared in Example 1 was put in a 300 mL four-necked flask equipped with a stirrer. After the liquid temperature was adjusted to 75° C., toluene (28.2 g) as a crystallization solvent 2 was added dropwise over 20 minutes. At this time, the liquid temperature decreased to 72° C. After 35 minutes elapsed while the temperature was maintained at 73° C., toluene (14.2 g) was further added dropwise over 15 minutes. After an elapse of 1 hour and 10 minutes since the dropwise addition, the liquid temperature was lowered to 50° C. at a rate of 10° C./h, after which the resultant was allowed to cool to 25° C. At this time, the precipitated crystal hardly adhered to the flask wall surface and was uniformly dispersed in a crystallization solution.

The precipitated crystal was separated by filtration using a centrifugal filter and washed with toluene (29.0 g). The obtained crystal was added to a 200 mL eggplant-shaped flask, and dried at 80° C. for 3 hours under a reduced pressure of 1.5 kPa to obtain a dried crystal of compound A.

The purity of compound A in the obtained crystal was 98.7 area %, and the yield was 71% in terms of 4,4′-diacetylbiphenyl.

The obtained crystal contained 0.8 wt % o-cresol, 0.6 wt % butyl acetate, and 14.5 wt % toluene.

As a result of DSC analysis of the obtained crystal, it was confirmed that the crystal had endothermic peak top temperatures of 125° C., 176° C., and 241° C. and was a crystal β. The DSC data are shown in FIG. 5.

Diffraction angles 2θ (°) and relative intensities of diffraction peaks appearing in a PXRD measurement of the obtained crystal are shown in Table 3 as in Example 1. The PXRD measurement chart is shown in FIG. 6.

TABLE 3

| | Relative |
|---|---|
| 2θ (°) | intensity |
| 8.9 | 20 |
| 11.1 | 44 |
| 13.4 | 100 |
| 15.9 | 52 |
| 16.4 | 62 |
| 17.4 | 34 |
| 17.9 | 46 |
| 19.2 | 40 |
| 19.9 | 33 |
| 21.6 | 35 |
| 22.5 | 31 |
| 24.2 | 37 |
| 24.5 | 37 |
| 27.8 | 25 |

### Example 4

A portion (61.8 g) of the preparation solution A prepared in Example 1 was put in a 300 mL four-necked flask equipped with a stirrer. After the liquid temperature was adjusted to 74° C., toluene (28.3 g) as a crystallization solvent 2 was added dropwise over 20 minutes. At this time, the liquid temperature decreased to 69° C. After 15 minutes elapsed while the temperature was maintained at 73° C., 80 mg of the crystal obtained in Example 3 was added as a seed crystal. After 20 minutes elapsed while the temperature was maintained at 73° C. after the addition, toluene (14.0 g) was further added dropwise over 15 minutes. After 10 minutes, 80 mg of the crystal obtained in Example 3 was further added. One hour after the addition, the liquid temperature was lowered to 50° C. at a rate of 10° C./h, after which the resultant was allowed to cool to 25° C. At this time, the precipitated crystal hardly adhered to the flask wall surface and was uniformly dispersed in a crystallization solution.

The precipitated crystal was separated by filtration using a centrifugal filter and washed with toluene (28.2 g). The obtained crystal was added to a 200 mL eggplant-shaped flask, and dried at 80° C. for 3 hours under a reduced pressure of 1.2 kPa to obtain a dried crystal of compound A.

The purity of compound A in the obtained crystal was 99.0 area %, and the yield was 74.0% in terms of 4,4′-diacetylbiphenyl.

The obtained crystal contained 0.3 wt % o-cresol, 0.3 wt % butyl acetate, and 14.4 wt % toluene.

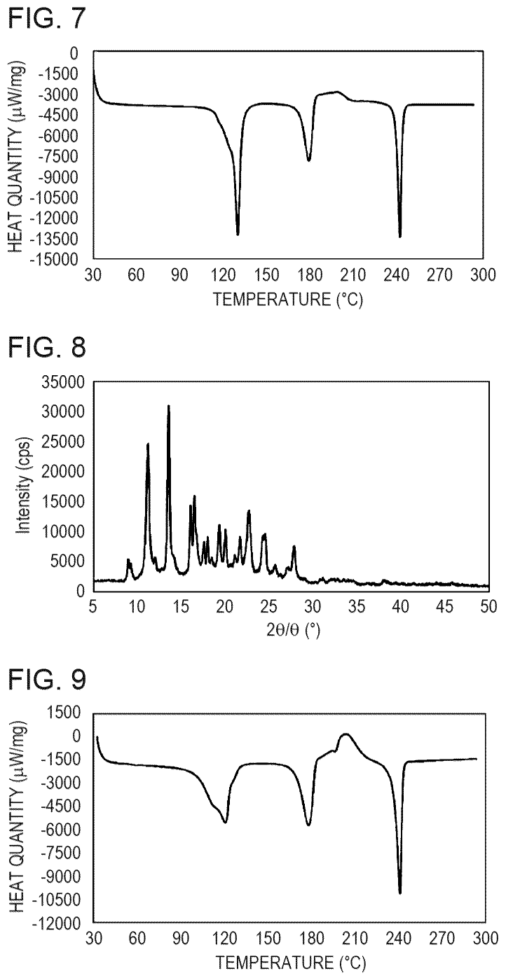

As a result of DSC analysis of the obtained crystal, it was confirmed that the crystal had endothermic peak top temperatures of 125° C., 178° C., and 242° C. and was a crystal β. The DSC data are shown in FIG. 7.

Diffraction angles 2θ (°) and relative intensities of diffraction peaks appearing in a PXRD measurement of the obtained crystal are shown in Table 4 as in Example 1. The PXRD measurement chart is shown in FIG. 8. These measurement data reveal that the crystal belongs to the same crystal system as the crystal obtained in Example 3.

TABLE 4

| | Relative |
|---|---|
| 2θ (°) | intensity |
| 11.2 | 80 |
| 13.5 | 100 |
| 16.0 | 46 |
| 16.5 | 51 |
| 17.5 | 27 |
| 18.0 | 29 |
| 19.3 | 36 |
| 20.0 | 33 |
| 21.0 | 20 |
| 21.6 | 29 |
| 22.7 | 43 |
| 24.2 | 30 |
| 24.5 | 31 |
| 27.8 | 24 |

### Example 5

A portion (61.2 g) of the preparation solution A prepared in Example 1 was put in a 300 mL four-necked flask equipped with a stirrer. After the liquid temperature was adjusted to 75° C., ethylbenzene (28.2 g) as a crystallization solvent 2 was added dropwise over 20 minutes. At this time, the liquid temperature decreased to 69° C. After stirring was performed for 35 minutes while the temperature was maintained at 72° C., ethylbenzene (14.2 g) was further added dropwise over 15 minutes. After 1 hour and 10 minutes, the liquid temperature was lowered to 50° C. at a rate of 10° C./h, after which the resultant was allowed to cool to 25° C. At this time, the precipitated crystal hardly adhered to the flask wall surface and was uniformly dispersed in a crystallization solution.

The precipitated crystal was separated by filtration using a centrifugal filter and washed with ethylbenzene (28.2 g). The obtained crystal was added to a 200 mL eggplant-shaped flask, and dried at 80° C. for 3 hours under a reduced pressure of 1.5 kPa to obtain a dried crystal of compound A.

The purity of compound A in the obtained crystal was 99.0 area %, and the yield was 60% in terms of 4,4′-diacetylbiphenyl.

The obtained crystal contained 0.8 wt % o-cresol, 0.9 wt % butyl acetate, and 15.9 wt % ethylbenzene.

As a result of DSC analysis of the obtained crystal, it was confirmed that the crystal had endothermic peak top temperatures of 121° C., 178° C., and 241° C. and was a crystal β. The DSC data are shown in FIG. 9.

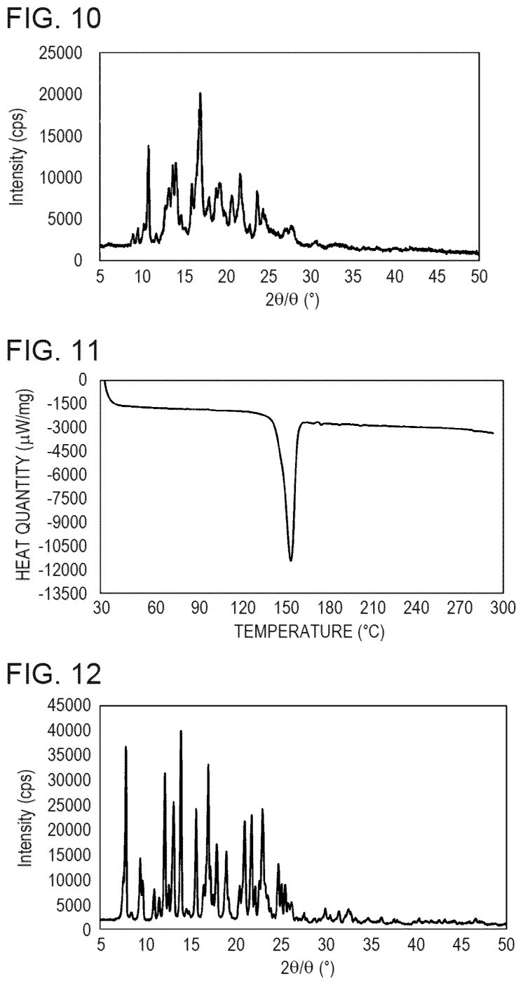

Diffraction angles 2θ (°) and relative intensities of diffraction peaks appearing in a PXRD measurement of the obtained crystal are shown in Table 5 as in Example 1. The PXRD measurement chart is shown in FIG. 10.

TABLE 5

| | Relative |
|---|---|
| 2θ (°) | intensity |
| 10.2 | 22 |
| 10.8 | 69 |
| 12.8 | 32 |
| 13.2 | 43 |
| 13.6 | 57 |
| 14.0 | 58 |
| 14.6 | 27 |
| 15.9 | 46 |
| 16.9 | 100 |
| 17.9 | 37 |
| 18.8 | 43 |
| 19.2 | 46 |
| 20.6 | 39 |
| 21.6 | 52 |
| 22.8 | 22 |
| 23.6 | 41 |
| 27.7 | 21 |

### Example 6

To a 5 L four-necked flask equipped with a stirrer, o-cresol (282.1 g), methanesulfonic acid (225.1 g), and dodecyl mercaptan (15.8 g) were added, and after nitrogen purging was performed, the temperature was raised to 50° C. While the temperature in the flask was maintained at 50° C., a solution of o-cresol (1171.6 g) and 4,4′-diacetylbiphenyl (310.1 g) adjusted to 50° C. was mixed with stirring over 5 hours. Thereafter, stirring was performed for 22 hours. After a 16% aqueous sodium hydroxide solution (624.2 g) and a 75% aqueous phosphoric acid solution (14.8 g) were added to achieve partial neutralization, the temperature was raised to 75° C. with butyl acetate (1651.2 g) added, and an aqueous layer was removed.

After water (828.4 g) was added into the flask to wash an oil layer with water, an aqueous layer was removed. Reduced-pressure distillation was performed with heating, and o-cresol, butyl acetate, and water were recovered until the distillation residue weighed 988.3 g. Thereafter, butyl acetate (811.9 g) was added as a crystallization solvent 1. After the liquid temperature was adjusted to 76° C., cyclohexane (814.9 g) as a crystallization solvent 2 was added dropwise over 20 minutes. At this time, the liquid temperature decreased to 68° C. After 10 minutes elapsed while the temperature was maintained at 73° C., 1.7 g of the crystal α of compound A obtained in Example 2 was added as a seed crystal. After 15 minutes elapsed while the temperature was maintained at 73° C. after the addition, cyclohexane (411.3 g) was further added dropwise over 15 minutes. After the resultant was held at 73° C. for 30 minutes, cyclohexane (641.9 g) was further added dropwise over 25 minutes. One hour after the addition, the liquid temperature was lowered to 59° C. at a rate of 10° C./h, after which the resultant was allowed to cool to 25° C. At this time, the precipitated crystal hardly adhered to the flask wall surface and was uniformly dispersed in a crystallization solution.

The precipitated crystal was separated by filtration using a centrifugal filter and washed with cyclohexane (816.0 g). The obtained crystal was transferred to a 2 L eggplant-shaped flask, and dried at 80° C. for 3 hours under a reduced pressure of 0.5 kPa to obtain a dried crystal of compound A.

The purity of compound A in the obtained crystal was 97.8 area %, and the yield was 67% in terms of 4,4′-diacetylbiphenyl.

The obtained crystal contained 0.5 wt % o-cresol, 3.8 wt % butyl acetate, and 7.8 wt % cyclohexane.

As a result of DSC analysis of the obtained crystal, it was confirmed from an endothermic peak top temperature that the crystal had a melting point of 157° C. and was a crystal α. The DSC data are shown in FIG. 11.

Diffraction angles 2θ (°) and relative intensities of diffraction peaks appearing in a PXRD measurement of the obtained crystal are shown in Table 6 as in Example 1. The PXRD measurement chart is shown in FIG. 12.

TABLE 6

| | Relative |
|---|---|
| 2θ (°) | intensity |
| 7.5 | 26 |
| 7.8 | 92 |
| 9.4 | 36 |
| 9.7 | 25 |
| 10.9 | 20 |
| 12.1 | 79 |
| 12.6 | 23 |
| 13.1 | 64 |
| 13.9 | 100 |
| 15.6 | 61 |
| 16.4 | 22 |
| 16.9 | 83 |
| 17.2 | 32 |
| 17.9 | 43 |
| 18.9 | 39 |
| 20.4 | 22 |
| 21.0 | 55 |
| 21.7 | 58 |
| 22.1 | 22 |
| 22.6 | 24 |
| 22.9 | 61 |
| 23.2 | 23 |
| 24.7 | 33 |
| 25.1 | 23 |
| 25.4 | 23 |

### Example 7

After the crystal (602.9 g) of compound A prepared in Example 6 and butyl acetate (1060.0 g) were added to a 3 L four-necked flask equipped with a stirrer, the temperature was raised to 70° C. After water was added into the flask to wash an oil layer with water, an aqueous layer was removed. After the water-washing of an oil layer and the removal of an aqueous layer was performed six times in total, reduced-pressure distillation was performed with heating, and o-cresol, butyl acetate, and water were recovered until the distillation residue weighed 1030.6 g.

After the liquid temperature was adjusted to 80° C., cyclohexane (259.9 g) as a crystallization solvent 2 was added dropwise over 20 minutes. At this time, the liquid temperature decreased to 75° C. The crystal α of compound A in an amount of 357 mg was added as a seed crystal, and after the resultant was held at 75° C. for 1 hour, cyclohexane (262.9 g) was further added dropwise over 25 minutes. After the resultant was held at 75° C. for 20 minutes, cyclohexane (524.2 g) was further added dropwise over 50 minutes. Thereafter, the resultant was allowed to cool to 25° C. At this time, the precipitated crystal hardly adhered to the flask wall surface and was uniformly dispersed in a crystallization solution.

The precipitated crystal was separated by filtration using a centrifugal filter and washed with cyclohexane (909.9 g). The obtained crystal was transferred to a 1 L eggplant-shaped flask, and dried at 80° C. for 3 hours under a reduced pressure of 1.1 kPa to obtain a dried crystal of compound A.

The purity of compound A in the obtained crystal was 99.2 area %, and the yield was 51% in terms of 4,4′-diacetylbiphenyl.

The obtained crystal contained 4.7 wt % butyl acetate and 7.6 wt % cyclohexane.

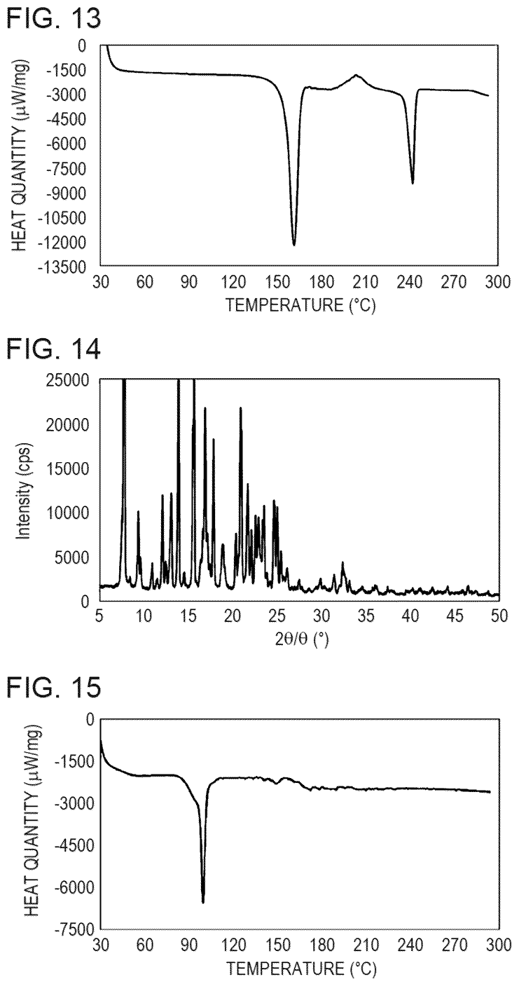

As a result of DSC analysis of the obtained crystal, it was confirmed from endothermic peak top temperatures that the crystal had melting points of 161° C. and 241° C. and was a crystal α. The DSC data are shown in FIG. 13.

Diffraction angles 2θ (°) and relative intensities of diffraction peaks appearing in a PXRD measurement of the obtained crystal are shown in Table 7 regarding peaks whose relative intensity based on a peak with the greatest intensity (measured intensity) is 10 or more. The PXRD measurement chart is shown in FIG. 14.

TABLE 7

| | Relative |
|---|---|
| 2θ (°) | intensity |
| 7.7 | 47 |
| 7.8 | 100 |
| 9.4 | 15 |
| 12.0 | 17 |
| 13.0 | 17 |
| 13.9 | 43 |
| 15.5 | 28 |
| 15.6 | 42 |
| 16.6 | 12 |
| 16.9 | 31 |
| 17.1 | 11 |
| 17.8 | 26 |
| 20.3 | 11 |
| 20.9 | 31 |
| 21.7 | 19 |
| 22.1 | 11 |
| 22.5 | 14 |
| 22.9 | 14 |
| 23.3 | 13 |
| 23.5 | 15 |
| 24.6 | 16 |
| 25.0 | 15 |

### Reference Example 1

A portion (61.6 g) of the preparation solution A prepared in Example 1 was put in a 300 mL four-necked flask equipped with a stirrer. After the liquid temperature was adjusted to 75° C., isooctane (27.9 g) as a crystallization solvent 2 was added dropwise over 20 minutes. At this time, the liquid temperature decreased to 72° C. The liquid in the flask was separated into two layers. After stirring was performed for 35 minutes while the temperature was maintained at 73° C., isooctane (14.1 g) was further added dropwise over 15 minutes. After the resultant was held at 73° C. for 1 hour, the liquid temperature was lowered to 50° C. at a rate of 10° C./h, after which the resultant was allowed to cool to 25° C. Most of the precipitated solid adhered to the flask wall surface to form masses. The adhering masses were peeled off with a spatula, and the masses were then separated by filtration using a centrifugal filter and washed with isooctane (28.4 g). When the masses in the flask were transferred to the centrifugal filter, events that would be very problematic in industrial production were observed; specifically, large masses were stuck in the neck of the flask, and some masses remained at the bottom of the flask. In addition, the presence of the masses prevented the crystal from uniformly attaching to a filter cloth of the centrifugal filter, so that the filtration operation could not be performed uniformly throughout.

The masses separated by filtration were transferred to a 200 mL eggplant-shaped flask, and dried at 80° C. for 3 hours under a reduced pressure of 1.5 kPa to obtain a dried crystal of compound A. The dried crystal had many masses.

The purity of compound A in the obtained crystal was 94.5 area %, and the yield was 91% in terms of 4,4′-diacetylbiphenyl.

The obtained crystal contained 5.3 wt % o-cresol, 2.8 wt % butyl acetate, and 3.6 wt % isooctane.

As a result of DSC analysis of the obtained crystal, the melting point was found to be 101° C. from an endothermic peak top. The DSC data are shown in FIG. 15.

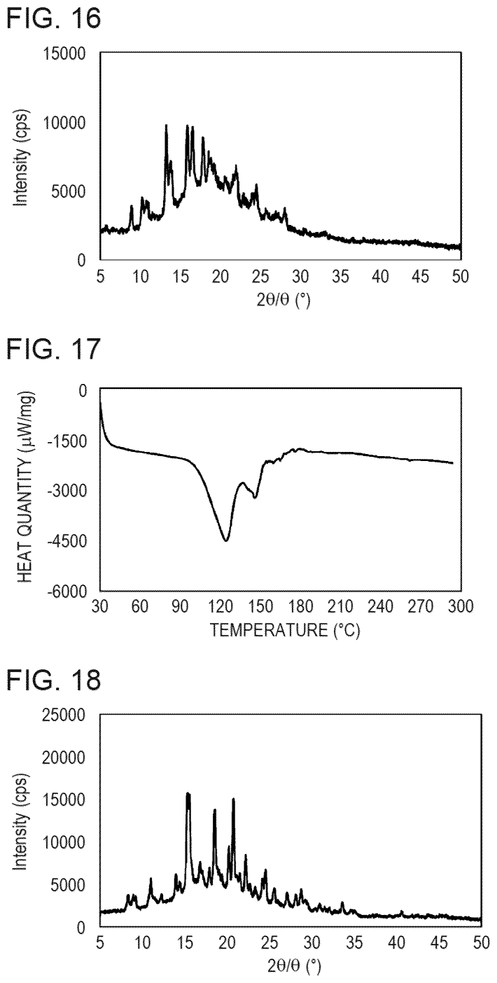

Diffraction angles 2θ (°) and relative intensities of diffraction peaks appearing in a PXRD measurement of the obtained crystal are shown in Table 8 as in Example 1. The PXRD measurement chart is shown in FIG. 16.

TABLE 8

| | Relative |
|---|---|
| 2θ (°) | intensity |
| 8.9 | 40 |
| 10.2 | 46 |
| 10.7 | 44 |
| 13.2 | 100 |
| 13.8 | 73 |
| 15.8 | 99 |
| 16.5 | 98 |
| 17.8 | 91 |
| 18.5 | 80 |
| 21.9 | 70 |
| 24.5 | 56 |

### Reference Example 2

A portion (63.3 g) of the preparation solution A prepared in Example 1 was put in a 300 mL four-necked flask equipped with a stirrer. After the liquid temperature was adjusted to 74° C., methanol (29.3 g) as a crystallization solvent 2 was added dropwise over 20 minutes. At this time, the liquid temperature decreased to 69° C. After stirring was performed for 35 minutes while the temperature was maintained at 69° C., methanol (14.2 g) was further added dropwise over 15 minutes. After the resultant was held at 69° C. for 1 hour, the liquid temperature was lowered to 50° C. at a rate of 10° C./h, after which the resultant was allowed to cool to 25° C. The precipitated crystal was separated by filtration using a centrifugal filter and washed with methanol (28.1 g). The crystal separated by filtration was muddy. When this crystal was transferred into a 200 mL eggplant-shaped flask, events that would be very problematic in industrial production were observed; specifically, for example, it was necessary to place a muddy filter residue on a funnel and push it into the flask using a spatula, and in addition, since a mass of the crystal adhered to the spatula during this operation, it was necessary to shake off the crystal by swinging the spatula.

The crystal in the eggplant-shaped flask was dried at 80° C. for 3 hours under a reduced pressure of 1.2 kPa to obtain a dried crystal of compound A. During the drying, melting of a portion of the crystal was observed.

The purity of compound A in the obtained crystal was 96.7 area %, and the yield was 80% in terms of 4,4′-diacetylbiphenyl.

The obtained crystal contained 2.7 wt % o-cresol, 0.6 wt % butyl acetate, and 4.3 wt % methanol.

As a result of DSC analysis of the obtained crystal, endothermic peak top temperatures were 124° C. and 146° C. The DSC data are shown in FIG. 17.

Diffraction angles 2θ (°) and relative intensities of diffraction peaks appearing in a PXRD measurement of the obtained crystal are shown in Table 9 as in Example 1. The PXRD measurement chart is shown in FIG. 18.

TABLE 9

| | Relative |
|---|---|
| 2θ (°) | intensity |
| 8.4 | 24 |
| 8.9 | 23 |
| 9.2 | 23 |
| 11.0 | 36 |
| 12.3 | 25 |
| 14.0 | 39 |
| 14.4 | 34 |
| 15.4 | 100 |
| 15.6 | 99 |
| 16.8 | 48 |
| 17.9 | 44 |
| 18.6 | 88 |
| 20.3 | 60 |
| 20.8 | 96 |
| 21.5 | 40 |
| 22.2 | 54 |
| 23.3 | 29 |
| 24.2 | 36 |
| 24.5 | 43 |
| 25.5 | 29 |
| 27.1 | 26 |
| 28.1 | 24 |
| 28.7 | 28 |

### <Bulk Density Measurement of Crystal>

The loose bulk density and the tapped bulk density of the crystals obtained in Examples 1 to 5 and 7 were measured by the above analysis methods.

The crystals obtained in Reference Examples 1 and 2 were in the form of masses at the time of crystal precipitation, filtration, and drying, and needed to be crushed with a spatula for handling. Therefore, the crystals were not in the form of uniform powder, and their measured bulk density values varied depending on the degree of crushing of the masses, so that a proper bulk density measurement could not be performed.

The crystallization conditions and the obtained crystals in Examples 1 to 5, Example 7, and Reference Examples 1 and 2 are listed in Table 10. In the “Bulk density” column in Table 10, “*” denotes that the measurement was not performed, and “-” denotes that the measurement could not be performed.

TABLE 10

| | | | | Crystal | | | | | | | | |
|---|---|---|---|---|---|---|---|---|---|---|---|---|
| | | | | | | DSC peak | | Solvent content | | | | |
| | Crystallization conditions | | | | | top | | (wt %) | | | | |
| | Crystal- | Crystal- | Use of | | | temper- | | | | Crystal- | Bulk density | |
| | lization | lization | seed | Crystalline | Crystal | ature | Purity | | Butyl | lization | (g/cm³) | |
| | solvent 1 | solvent 2 | crystal | form | system | (° C.) | (LC area %) | o-Cresol | acetate | solvent 2 | Loose | Tapped |
| Example | butyl | cyclohexane | no | powder | crystal α | 157 | 98.1 | 0.8 | 2.4 | 7.4 | 0.38 | 0.52 |
| 1 | acetate | | | | | | | | | | | |
| Example | | | yes | powder | | 157 | 98.5 | 0.4 | 2.2 | 7.8 | 0.38 | 0.50 |
| 2 | | | | | | | | | | | | |
| Example | | toluene | no | powder | crystal β | 125 | 98.7 | 0.8 | 0.6 | 14.5 | 0.36 | 0.53 |
| 3 | | | | | | 176 | | | | | | |
| | | | | | | 241 | | | | | | |
| Example | | | yes | powder | | 125 | 99.0 | 0.3 | 0.3 | 14.4 | 0.33 | 0.54 |
| 4 | | | | | | 178 | | | | | | |
| | | | | | | 242 | | | | | | |
| Example | | ethylbenzene | no | powder | | 121 | 99.0 | 0.8 | 0.9 | 15.9 | 0.32 | 0.41 |
| 5 | | | | | | 178 | | | | | | |
| | | | | | | 241 | | | | | | |
| Example | | cyclohexane | yes | powder | crystal α | 157 | 97.8 | 0.5 | 3.8 | 7.8 | * | * |
| 6 | | | | | | | | | | | | |
| Example | | | yes | powder | | 161 | 99.2 | 0 | 4.7 | 7.6 | 0.38 | 0.50 |
| 7 | | | | | | 241 | | | | | | |
| Reference | | isooctane | no | massive | — | 101 | 94.5 | 5.3 | 2.8 | 3.6 | — | — |
| Example | | | | solid | | | | | | | | |
| 1 | | | | | | | | | | | | |
| Reference | | methanol | no | muddy | — | 124 | 96.7 | 2.7 | 0.6 | 4.3 | — | — |
| Example | | | | solid | | 146 | | | | | | |
| 2 | | | | | | | | | | | | |

When crystallization was performed using the butyl acetate-isooctane mixed solvent in Reference Example 1, events that would be very problematic in industrial production were observed; specifically, for example, the liquid was separated into two layers when the solvents were mixed, most of the precipitated solid adhered to the wall surface of the flask and needed to be peeled off with a spatula, the solid had many masses and was difficult to transfer, and the solid was not uniformly attached to a filter cloth of the centrifugal filter because of the many masses.

Failure to be uniformly attached to the filter cloth of the centrifugal filter leads to ununiform passage of a filtrate and ununiform pouring of a washing solvent over a filter residue, resulting in crystals with quality variations, leads to ununiform rotation in centrifugal filtration to cause vibration and noise, and leads to a strain on the shaft of the filter, thus increasing the risk of breakage. In large-scale manufacturing such as industrial production, these problems become more pronounced, and vibration and noise may present environmental problems around manufacturing facilities. Therefore, the crystal is considered to be unsuitable for industrial production.

When crystallization was performed using the butyl acetate-methanol mixed solvent in Reference Example 2, the filter residue was muddy. Therefore, the amount of the solvent contained in the filter residue is very large, and workers who handle the filter residue are exposed to a large amount of the solvent and become more likely to suffer from health problems. In addition, there was also the problem of difficulty in transferring as described above. Therefore, the crystal is considered to be unsuitable for industrial production as in the case of the method of Reference Example 1.

When crystallization was performed using the butyl acetate-cyclohexane mixed solvent in Examples 1 and 2 and Examples 6 and 7, the butyl acetate-toluene mixed solvent in Examples 3 and 4, or the butyl acetate-ethylbenzene mixed solvent in Example 5, which are specific examples of the method for producing the crystal according to the present invention, the problems encountered in Reference Examples 1 and 2 were not observed.

Specifically, the precipitated crystal hardly adhered to the wall surface of the flask and was uniformly dispersed in the crystallization solution. In the filtration operation, the crystallization solution was smoothly transferred to the filter, and the filter residue was uniformly attached to the filter cloth. The filter residue contained the solvent in a small amount, and had easy-to-handle properties so as not to adhere to the spatula.

From these, it has become clear that according to the production method of the present invention, compound A can be isolated as a crystal, and the crystal of compound A can be efficiently obtained also in industrial production.

Furthermore, when compared in terms of purity, the crystals obtained by the production method according to the present invention are 1.4 to 4.5 area % higher than the crystals obtained by the production methods in Reference Examples 1 and 2, and it has also become clear that compound A with high purity can be obtained.

As shown by the results of Examples 1 and 2 and Examples 3 and 4, it has become clear that adding a seed crystal enables production of a crystal with higher purity.

1. A crystal of 4,4′-bis(1,1-bis(4-hydroxy-3-methylphenyl)ethyl)biphenyl.

2. The crystal of 4,4′-bis(1,1-bis(4-hydroxy-3-methylphenyl)ethyl)biphenyl according to claim 1, wherein an endothermic peak top temperature determined by differential scanning calorimetry is in a range of 150° C. to 165° C.

3. The crystal of 4,4′-bis(1,1-bis(4-hydroxy-3-methylphenyl)ethyl)biphenyl according to claim 1, having diffraction peaks at diffraction angles 2θ of 7.8°=0.2°, 13.8°±0.2°, and 16.9°±0.2° in a powder X-ray diffraction peak pattern obtained using Cu-Kα radiation.

4. A method for producing the crystal of 4,4′-bis(1,1-bis(4-hydroxy-3-methylphenyl)ethyl)biphenyl according to claim 1, the method comprising a step of crystallizing 4,4′-bis(1,1-bis(4-hydroxy-3-methylphenyl)ethyl)biphenyl from a solution containing an alkyl acetate ester solvent having a total of 4 to 8 carbon atoms and a cyclic saturated hydrocarbon solvent having 5 to 8 carbon atoms.

5. The crystal of 4,4′-bis(1,1-bis(4-hydroxy-3-methylphenyl)ethyl)biphenyl according to claim 1, wherein endothermic peak top temperatures determined by differential scanning calorimetry are in a range of 120° C. to 130° C., a range of 170° C. to 185° C., and a range of 235° C. to 245° C.

6. The crystal of 4,4′-bis(1,1-bis(4-hydroxy-3-methylphenyl)ethyl)biphenyl according to claim 1, having diffraction peaks at diffraction angles 2θ of 11.1°±0.2° and 13.4°±0.2° in a powder X-ray diffraction peak pattern obtained using Cu-Kα radiation.

7. The crystal of 4,4′-bis(1,1-bis(4-hydroxy-3-methylphenyl)ethyl)biphenyl according to claim 1, having diffraction peaks at diffraction angles 2θ of 10.8°±0.2° and 16.9°±0.2° in a powder X-ray diffraction peak pattern obtained using Cu-Kα radiation.

8. A method for producing the crystal of 4,4′-bis(1,1-bis(4-hydroxy-3-methylphenyl)ethyl)biphenyl according to claim 1, the method comprising a step of crystallizing 4,4′-bis(1,1-bis(4-hydroxy-3-methylphenyl)ethyl)biphenyl from a solution containing an alkyl acetate ester solvent-based solvent having a total of 4 to 8 carbon atoms and an aromatic hydrocarbon solvent having 7 to 10 carbon atoms.

9. A method for producing the crystal of 4,4′-bis(1,1-bis(4-hydroxy-3-methylphenyl)ethyl)biphenyl according to claim 2, the method comprising a step of crystallizing 4,4′-bis(1,1-bis(4-hydroxy-3-methylphenyl)ethyl)biphenyl from a solution containing an alkyl acetate ester solvent having a total of 4 to 8 carbon atoms and a cyclic saturated hydrocarbon solvent having 5 to 8 carbon atoms.

10. A method for producing the crystal of 4,4′-bis(1,1-bis(4-hydroxy-3-methylphenyl)ethyl)biphenyl according to claim 5, the method comprising a step of crystallizing 4,4′-bis(1,1-bis(4-hydroxy-3-methylphenyl)ethyl)biphenyl from a solution containing an alkyl acetate ester solvent-based solvent having a total of 4 to 8 carbon atoms and an aromatic hydrocarbon solvent having 7 to 10 carbon atoms.
